(19) United States
(12) Patent Application Publication
(10) Pub. No.: US 2007/0208090 A1
(43) Pub. Date: Sep. 6, 2007

(54) Method for Converting Natural Gas Into Synthesis Gas for Further Conversion Into Organic Liquids or Methanol and/or Dimethyl Ether

(75) Inventor: Christiaan P. van Dijk, Houston, TX (US)

Correspondence Address: AKIN GUMP STRAUSS HAUER & FELD LLP, 1111 Louisiana Street, 44th Floor, Houston, TX, 77002, US

(73) Assignee: VAN DIJK TECHNOLOGIES, L.LC., Houston, TX (US)

(21) Appl. No.: 11/629,713

(22) PCT Filed: Jun. 20, 2005
(86) PCT No.: PCT/US05/21920

§ 371 Date: Dec. 14, 2006

Related U.S. Application Data
(60) Provisional application No. 60/583,669, filed on Jun. 29, 2004.

(51) Int. Cl. C07C 27/06

(52) U.S. Cl. 518/702; 518/726

(57) ABSTRACT

The APPLICATION protects the production of a plurality of vessel-based gas-to liquids plants, at least identical in the conversion of natural gas into synthesis gas. The vessel can be a ship or a barge. The liquids made can be either hydrocarbons, made under Fischer-Tropsch type conditions, or oxygen-containing methanol or dimethyl-ether The given approach offers the possibility for a substantial income possibility, even at substantially increased natural gas costs.

### 1. FIELD OF THE INVENTION

This invention pertains to methods for converting natural gas into synthesis gas, this for further conversion into organic liquids and/or methanol and/or dimethyl ether.

### 2. DESCRIPTION OF THE RELATED ART

Chemical plants are commonly built at a site chosen for the production and sale or use of the product. Recently it has been proposed to build chemical plants on vessels, such as ships or barges. The new facility can be built at an existing construction site with all the background of personnel and equipment, which then is continuously available. Generally a considerable amount of storage comes for free with use of a vessel. After construction the plant can be transported over water to its operating site and after product production service there the plant can be transported elsewhere. Use of a ship- or barge-based production facility is of great advantage, when use is made of natural gas, produced far away from shore. Expensive transport of gas to shore and possibly also of produced fluids from shore to a loading point then can be avoided. The only important difference with normal shore-based plants is that use of liquefied oxygen is not allowed in ship-or barge-type operation, due to safety considerations.

Normally chemical plants are built at the site where future production is to be made. Further, in a particular market, any operating Company will only build one new plant at the same time. This practice has developed from the economical necessity of operating the plants, after construction, as soon as possible at a close approach to full capacity. Especially when introducing a new product the market response decides the size of the extra production. But also when providing new production capacity in an existing market it is often unattractive from marketing reasons to start the new plant at maximum capacity. Because it normally takes time for the market to adjust to allow full use of one new plant's capacity, it stands to reason, that building two identical plants in the same market aggravates the problem unnecessarily.

A different approach is, however, now proposed, when the unusual situation is encountered, where the market expanse is predictably already present, as soon as the new production has become a fact. This case is encountered in the growing necessity to use in an attractive manner the large amounts of natural gas, which are found and can be produced in areas far away from the developed parts of the world. The gas sources in the developed areas of the world are being used intensively and therefore that gas is of high value. The gas in the undeveloped areas is often called stranded gas, indicating the large difference in value with gas sources in the industrial developed areas.

This large difference in values of the different streams of natural gas has resulted in a large driving force towards some accepted use of the stranded gas. Several approaches have been proposed and one of them is being practiced already on a substantial scale. This concerns the Liquefaction of the Natural Gas (LNG), followed by transport of this liquid to more developed areas, where after evaporation under pressure the gas is fed into the existing pipeline structure. A very large capital expenditure is necessary to achieve the total of these steps. Also, because of the large size of any LNG production, necessary for achieving acceptable returns on investment, only the largest of natural gas sources can be used for this purpose. Notwithstanding the disadvantages, the result is a considerable increase in value of the natural gas involved in this operation. Most owners of such large gas fields (with potential use as LNG source) therefore assess the value of that gas at substantial higher value than that of fields too small to support a LNG facility. This is the case, notwithstanding the fact, that the lack of sufficient delivery stations for evaporation of the LNG into existing pipelines has precluded a number of LNG proposals to materialize.

A substantial number of different types of conversions of natural gas to liquid products are expected to result in the near future, this from the need of expanding the amount of hydrocarbon liquids that the developed world is consuming in larger and larger amounts, while crude oil production is not able to keep up with the rate of growth of the market demands. Several new developments are based on first converting the gas into a Synthesis Gas, containing both carbon oxides and hydrogen in different ratios, followed by conversion of such synthesis gas into hydrocarbon liquids (Fischer-Tropsch type reactions). Next to this also the conversion of the Synthesis Gas on large scale into methanol and/or dimethyl ether has been proposed, for which conversion a different catalyst is necessary and different conditions are being used. In order to keep the capital cost per unit of product as low as possible, the new proposals for manufacturing plants for these different operations are at very large scale, leading to preferred use of very large gas fields. These proposals therefore are aiming to a degree at similar large production sites for natural gas as the LNG proposals in order to provide the large amounts of gas necessary for these conversions. This competition for use of some of these large natural gas sources will only increase the estimated value difference between gas from these very large fields against that from the medium- to smaller-sized gas fields.

It should be noted, that the scale on which the conversion of natural gas to organic liquids (GTL) (including hydrocarbon liquids and methanol and/or dimethyl ether), which has to take place in the near future, will be very large. This is necessary because these products, and especially of these the organic liquids, will be used to compensate for the shortfall of crude oil production against its current demand world-wide. The standard approach consists of building very large plants, obtained by scaling up the different plant design of smaller scale existing today. To erect sufficient number of such large plants to satisfy the market demands would call for a very large investment of at least several hundred million dollars. The size of these investments clearly will be of major concern.

### BRIEF SUMMARY OF THE INVENTION

The present Application proposes a new and different approach for use of stranded gas, which would greatly diminish the need for providing very large amounts of capital for the construction of the plants for organic liquids and/or methanol or dimethyl ether. It also allows use of the smaller but still readily available gas sources, and is in view of the existing practice unexpected and surprising.

The starting point for the present Invention is the known tendency to move to gas conversion plants based on ships or barges. One of the aspects for this preference has already been indicated, namely that this allows construction of the conversion plant close to known centers for plant construction, thus profiting from the background in personnel and technical facilities, which then becomes readily available. Surprisingly it now has been realized, that when aiming at a very large scale of conversion of natural gas into Synthesis Gas for further reaction into organic liquids and/or methanol and/or dimethyl ether (DME), by necessity using a large array of natural gas producing sources, it is advantageous when planning to build the facilities on ships or barges to use at least a large number of identically designed conversion plants of one small size. This is an unusual and unexpected aberration of the common practice in developing conversion plants for production of large amounts of products, which is to move to larger and larger size of the plants involved. This, as has been practiced by chemical engineers routinely, will allow profiting from the rule that the capital cost of plants per unit of product is generally lower the larger the production size of the plant. The relationship involved is, that the plant cost goes up and down with the power of the production size ratio, with a power factor generally less than 1, and often as low as 0.6. Normally this is only true to a limited maximum size. Beyond this point it becomes necessary to use parallel plants.

Further, it generally is against established practice to build a number of identical plants simultaneously, because the market expansion for the new capacity takes time to develop. The logic there is that violating this rule would result in a longer drawn-out period of production, i.e., (pay back of capital) at less than full capacity. However, when the very unusual circumstance happens, that the market is ready to absorb a large to very large influx of new capacity, the new approach from the present Invention offers large incentives. This unusual aspect is expected for the future production of both liquid hydrocarbons and methanol and/or dimethyl ether (DME) from natural gas. As far as the hydrocarbons are concerned, the production of crude oil is falling behind the growth of its use and it is expected that in the near future crude oil production will even diminish. If the consumption of the liquid products made from crude oil is not diminished strongly, a very large production of liquids from natural gas will become necessary to satisfy the market demands.

The necessity for producing large amounts of methanol and/or DME is less generally realized. The possible large scale use of these chemicals in the conversion to ethylene and propylene is likely to replace, to a large degree, the more expensive ethane cracking and will therefore become necessary on a rather substantial scale. Further, methanol can be used in electrical cars, presently being developed, this to provide electricity from an easily stored liquid, which may be preferable over the use of pressurized hydrogen. Finally, if made at sufficiently low cost, methanol could be used to generate DME as diesel fuel, or even used as gasoline component or to be converted into a useful liquid gasoline hydrocarbon by reaction over ZSM-5. At a sufficiently still lower cost even use of methanol as fuel for gas turbines might become attractive.

It can therefore be safely predicted, that on a large scale Synthesis Gas will be made out of natural gas, which Synthesis Gas then can be converted by Fischer-Tropsch techniques into lower or higher liquid hydrocarbons, and/or with use of different conditions and different catalysts, be reacted into methanol or DME. In this Application both process forms will be grouped together and indicated by the term GTL processes (therefore processes to convert natural gas into organic liquids, be these liquids hydrocarbons as when Fischer-Tropsch conditions are used or methanol or dimethyl ether, when conditions for formation of these compounds are practiced).

As the super-big gas fields may be partly or totally reserved to the attractive production of LNG, the new production process of this invention preferably might use smaller gas fields, from which the gas is likely to be available at a lower relative cost.

Instead of building different GTL conversion plants, each GTL plant sized to suit a different particular field, it is advocated to use a large number of smaller and identical GTL conversion units, built on a vessel, such as ships or barges, using for each field the optimal number of conversion units. A first already considerable saving is obtainable by building such GTL plants as single units, each one at a different site. The use of the same design for these plants, i.e., GTL units, then results in a direct reduction of the cost of design. For example, if the cost of design is taken at 33% of total cost, then the building of 10 identically designed plants at 10 different sites reduces the cost of design of each of the individual plants, or production units, to 3.3% of the original one plant cost, thus reducing the total plant cost of each of the individual production units from 100% down to 100−33+3.3=70.3% of the original cost. Obviously a greater cost reduction can be obtained by using the construction of the plants on vessels, such as ships or barges, to change production of the plants the in series type only, building them at one, or perhaps a few, preferred central site or sites. In view of the large total number of plants being necessary, the cost reduction can be substantial, if plant construction cost would follow the well-known rule established in a number of completely unrelated fields, which states, that the reduction factor of the cost per unit for two different rates of production is twice the 10-based logarithm of the ratio of the number of entities produced per time unit. This cost reduction is simplified by saying, that for every increase by a multiple of 10 the cost per unit goes down by a factor of 2. This cost reduction can easily be much larger than the cost reduction obtained by size increases. It is expected, that with reasonably large numbers of small plants a cost reduction by a factor of 2 over that of the largest plants possibly made by scale-up can be reached, as long as the construction of these small plants, i.e., individual production units, is carried out in continuous manufacture, imitating a production line. As soon as produced, the plants on ships or barges will have to be moved to the gas fields to start their production of the desired products. The short time of construction of such production units brings with it another sizable advantage. Especially with very large plants, as mentioned earlier, construction time can easily amount to 2 to 4 years. This translates for one big plant to spending part of the construction capital cost early, which results in long periods of no return for some of the capital spent.

Apart from the cost reduction, as such, the use of ships or barges as the vessel containing the GTL production unit allows changing the number of production facilities in aging gas fields, when the natural gas production is diminishing, thus producing the desired products at closer to optimal loading as far as the conversion GTL units are concerned.

Use of ship- or barge-based GTL production units is especially of advantage, when the gas field is situated in deep water, far away from shore. The proposed operation then eliminates the need for transporting the gas to the shore, for which the pipeline might be very expensive, and also allows direct transfer of liquids produced, again without the possible need to construct a sea base for transfer of the liquids made into the large transport vessels.

An unexpected advantage of the switch to an array of a multiplicity of small plants, i.e., GTL production units, is that the size of the plants allows use of compact fired reformers, as proposed by several Companies, of which can be named Aker-Kvaerner and the combination of British Petroleum and Davy Process Technology. The possibility for producing with these new heat-exchange reformers not only Fischer-Tropsch type hydrocarbons, but also methanol is also pointed out in the article written by BP and Davy. These compact fired reformers do not scale very well and therefore so far have only been used in relatively small plants within the size limits chosen for the conversion plants, i.e., GTL units, as defined in this Application. The compact fired reformers offer the advantage of lower cost than plant designs based on adiabatic reforming with oxygen or oxygen-enriched air, because of the substantial costs connected with obtaining highly concentrated oxygen. These compact fired reformer plants are also likely to have lower energy use, thus diminishing the CO₂ production in the conversion of natural gas into organic liquids or either methanol and/or DME.

### DETAILED DESCRIPTION OF THE INVENTION

The advantages of the Invention are likely to increase with the size of the total production per time unit, as this results in the tendency to reduce the cost per unit. The best demonstration of the value of the Invention therefore is in the case of a relatively small market, for which the future advantage of the Invention was first understood.

The inventor believes, and this is assumed for sake of argument, that in the near future it will be attractive to convert methanol into ethylene and propylene at a very high combined selectivity, while the ratio of ethylene to propylene can within reason be varied at will. As the expected variation of the methanol to olefin (MTO) process will be in the fixed bed mode, it can also be expected, that a revamp of existing plants can be achieved at low cost. These plants now are producing both ethylene and propylene in somewhat varying amounts by thermally decomposing mixtures of ethane and higher hydrocarbons. As these plants presently are suffering from the very large cost increases of ethane or any other hydrocarbons used, the switch to methanol, which can be made at low cost from stranded gas, might well be highly attractive. In evaluating this still future possibility it became clear, that the capital cost for the methanol production from natural gas would be substantially higher than the cost of the revamp, thus resulting in a high total capital cost, which would present a barrier against fast acceptance.

Taking the ethylene production in the United States, of which close to 13.6 billion kg (30 billion pounds) per year is made by thermal cracking of ethane, conversion to methanol as feedstock would expect to demand circa 69 billion pounds per year, or circa 90,000 metric tons per day (MTPD) of methanol. These amounts correspond to the expected mixtures of ethylene and propylene necessary to imitate the presently existing production patterns. Following the present proposal towards ship- or barge-based plants, use of oxygen, made by liquefaction, is not acceptable from safety aspects. This leaves for design either adiabatic reforming with enriched oxygen, as presently still proposed by Starchem, or use of fired reforming, as long as a design is used with compact reformer structure, as proposed by the combination of BP-Davy and also by Aker-Kvaerner.

Instead of starting from a small number of large plants, taken at about 12,000 MTPD each, an alternate was investigated of about 30 identical plants, each of a capacity of slightly above 3,000 MTPD. The choice of this size small plant was arbitrary, and other choices might be preferred because of circumstances. However, this first choice serves well to show the impact of the Invention. Assume the basic cost for such a single 3,000 MTPD plant to be $350 million. Such smaller plants would be able to be fed by medium- or smaller-sized gas sources with a much less competitive gas price structure. If instead the production of methanol is assumed through the use of large expanded methanol plants, say of a size of 12,000 MTPD, which increase is likely to be with a power function of about 0.7, a capital cost is obtained of 350 million×4 to the power 0.7=$923.7 million. The assumption of only using these very large plants is optimistic, because with varying field size some smaller size might also have to be used. The cost reduction in ordering 30 plants will be ruled by the generally recognized reduction factor of 2 for every factor of 10, or by an actual factor of 2.95, leading to a cost per plant of $118.5 million. To arrive at a 12,000 MTPD capacity four of the 3,000 MTPD plants should be used in combined service, leading to a cost of 4 times $118.5, or $473.9 million, which is 1.95 times smaller. The capital cost for the smaller plants, making the same amount of methanol, will be significantly lower than the cost of the set of large methanol plants.

Already, when only taking the case of 10 small plants, the cost per plant then will be $175 MM, still allowing a significant advantage in capital in comparison to the 12,000 MTPD plant, next to the other advantages of the smaller plants. Obviously the advantages increase, when the number of identical units is increased beyond 20, or better beyond 50.

3,000 MTPD plants have been built a number of times, are proven out and relatively risk-free, while the scale-up to a maximum of 12,000 MTPD carries with it the risk, that unexpectedly some negatives show up. The further big advantage for the set of small plants is the possibility of using lower-cost gas from smaller gas sources, for which presently no other use can be indicated. Also, especially with mid-sized fields, the initial number of 3,000 MTPD methanol plants operating on the natural gas produced by that field can be diminished in line with the decreasing production of the field.

Further, it is indicated to use for the 3,000 MTPD methanol plants the designs for fired reformers with compact structure, which use of fired reforming leads to lower energy use.

For the 12,000 MTPD size use of the compact reformer might perhaps not be possible, due to the limited ship or barge size. Maintenance is also easy, as all the plants have identical parts. Finally, the construction in series is likely to lead to faster delivery of the individual plants. A higher estimate for the market can easily be accommodated through an increase in the size of the production line, further lowering the individual costs.

It does not have to be argued, that additional methanol plants can be simultaneously ordered, for instance for market study for other applications, thus further reducing the individual costs. This could help to explore at low capital cost uses of methanol for diesel fuel (dimethyl ether has been proven to be an almost ideal Diesel fuel) or use for fuel or for the electricity generation in electrical cars. Even making gasoline can be tested, perhaps after some necessary modification of the Mobil Oil plant design.

After these alternate uses are established, the much larger uses thus generated, will allow ordering of larger quantities of small plants, at still further reduced capital cost.

These advantages are not available at any size of the small plants. For instance the extra cost in personnel of separately operating a large number of plants makes it unattractive to lower the size too much. It is felt, that at a minimum the size of the Synthesis Gas production should be such, that at least 1,000 MTPD of Fischer-Tropsch type liquids or of methanol or equivalent amount of DME will be produced. Also, the maximum flexibility in the use of smaller fields is a strong factor against making the size of the plants too large. A maximum size of six times the given minimum value, i.e., 6,000 MTPD, is felt to be the limit.

A similar analysis indicates lower cost for the Fischer-Tropsch approach, when switching to more than 10 small plants. However, there is no easy early start, as possible with methanol in the expected application in revamping MTO processes before methanol is generated for other super-large scale applications. For the Fischer-Tropsch application the initial involvement by necessity has to be substantial in size, as the market will not be tempted by the availability of much smaller amounts than are necessary to attract the public. Also here the advantages increase, when a higher number of plants, like more than 20 or even more than 50, are manufactured in series.

It may be desirable to increase the fit between a desired production from a smaller-sized field and the set of smaller conversion plants. Then a small number of intermediate sized convertor plants might be desired. One could make a set of 3,000 MTPD methanol plants much more adaptable by also ordering a small number of 1,500 or even 4,500 MTPD plants. These plants are then less economic in comparison to the 3,000 MTPD variety, but the extra flexibility might be attractive.

This difficulty for obtaining the lowest-cost for the Fischer-Tropsch conversion plants can be overcome, when the reactor or reactors, responsible for making the final liquids, can be changed in design, so that at will plants can be manufactured for making methanol or Fischer-Tropsch liquids. For Fischer-Tropsch applications the reactor conditions for the conversion of the Synthesis Gas are different than for methanol, so that the result will be, that a number of the ship- or barge-based conversion plants can be used for methanol and/or DME, while the others will be used for Fischer-Tropsch type conversions. This still will result with a lower cost for the Fischer-Tropsch plants, but not as much as when a large number of these plants are constructed on one design only.

To demonstrate that further, if the design for the reformer is suited for both applications, then consider a combination of 10 plants for methanol and 10 plants for Fischer-Tropsch. With a similar ship or barge plus a similar front end of the plant the cost savings on that part of the total construction of the 20 plants is influenced by the number 20. The tail end of both plants, however, is only reduced by the effect of the number 10. The overall saving thus is larger than when both sets were constructed independent of one another.

While the above clearly demonstrates the advantages resulting from simultaneously ordering an array of ship- or barge-built plants, containing a large number of identical units, the actual size chosen for such identical units is still open and can depend on a number of factors, like readily availability of critical equipment, preferred size of the supporting vessels, or size of the gas fields considered, and on possibly other factors. This does not change the strength of the Invention in lowering the capital cost of the equipment involved, when a larger number of units can be ordered simultaneously.

The above rather complicated possibilities can best be explained by the following Examples.

The first Example demonstrates the economic advantage of large-scale manufacture of the Fischer-Tropsch products. In this Example the conditions are chosen to be more conservative than can be expected, this to not only provide more confidence, but also to respond to the possibility, that in the initial learning period of operation the different factors, necessary to obtain a smooth operation of the production in series, are not all at optimum condition.

Example [A] demonstrates how a production of plants, providing the conversion of low-cost gas into organic liquids, can start with a rather limited first capital, while still allowing a fast growth of the net income generated by the different plants. This is caused by the capital generated by the plants in continued operation, thus providing new capital for the new plants build in following years. This proves, that the operation as proposed in this PATENT APPLICATION eliminates the need for very large initial capital involvement.

The basis for Example [A] is the cost figure given in the article of J. Font Freide, B P, London, UK, Gamlin and M. Ashley, both from Davy Process Technology, London, UK, published in Hydrocarbon Processing of February 2003, pages 53 to 58. On page 58 it is stated, that construction of super-large portions of the plant, (probably, if that is allowable for transport to the construction site) tend to reduce the cost of construction considerably. This is expected to lead to a cost of $23,000 per daily barrel for the 17,000 barrels per day (BPD) plant under consideration in the article. This remark is of great value for the present Invention. The best construction of the ships or barges used, is possibly the technique followed by Henry Kaiser in WW II, when he demanded construction of his Liberty ships in sections, made by smaller shipyards, followed by welding the sections together in the final central yard. The different large pieces of the plant could for instance each be in one of those sections of ships or barges. The transport to the final shipyard would obviously be over water, whereupon the final yard would make the final connections both for plant and vessel.

The cost reduction following the construction of the plants in series, is taken at 50%, after assuming (probably conservatively) an added cost for the vessel and the plant of 20% over plant cost alone. The final cost thus becomes $23,000×1.20×0.5=$13,800 per daily barrel.

The crude oil price over the 11-year of the planned operation in Example [A] is taken at a steady $40/B, while the industry's expectation of that price is for an overall continual increase, starting from a considerably higher value at the time of writing this Application.

The value of the highly pure liquids, obtained by the BP-Davy variation of the Fischer-Tropsch process, is taken at 4/3 times the value of the crude oil.

The cost of the gas used from the smaller gas fields, together called “really stranded gas,” is taken at $2.50 per barrel product.

The capital draw, if needed at the end of an operating year, is penalized with a 15% fee interest on loan. A negative capital draw indicates ready net capital in hand.

A taxation is assumed of 35%, calculated on gross earnings minus 10% of capital operating and minus the fee on capital draw.

All numbers on costs are given in billion dollars per year.

### EXAMPLE [A]

| | YEAR | | | | | | | | | | |
|---|---|---|---|---|---|---|---|---|---|---|---|
| | 1 | 2 | 3 | 4 | 5 | 6 | 7 | 8 | 9 | 10 | 11 |
| Number of plants | 40 | 58 | 100 | 100 | 100 | 100 | 100 | 100 | 100 | 100 | 0 |
| PLANT COST | 9.4 | 13.6 | 23.5 | 23.5 | 23.5 | 23.5 | 23.5 | 23.5 | 23.5 | 23.5. | 0 |
| Billion $ PROD. | 6.3 | 21.6 | 46.3 | 77.6 | 108.9 | 140.1 | 171.4 | 202.7 | 234.0 | 265.3 | 280.9 |
| Billion $ FEED | 0.3 | 1.0 | 2.2 | 3.6 | 5.1 | 6.6 | 8.0 | 9.5 | 11.0 | 12.4 | 13.2 |
| 10% PLT CAP | 0.9 | 2.3 | 4.6 | 7.0 | 9.3 | 11.7 | 14.0 | 16.4 | 18.7 | 21.1 | 20.1 |
| 15% DRAW | 0.9 | 0.9 | 0 | 0 | 0 | 0 | 0 | 0 | 0 | 0 | 0 |
| 35% TAX | 1.5 | 6.1 | 13.8 | 23.4 | 33.0 | 42.7 | 52.3 | 61.9 | 71.5 | 81.1 | 86.7 |
| CAP DRAW | 5.7 | 5.7 | −1.1 | −28.2 | −75.4 | −142.9 | −230.5 | −338.4 | −466.4 | −614.6 | −795.7 |

After 10 years with all 898 plants paid for and no capital debt left, the total money in hand amounts to 614.6 billion dollars. This high income might also allow paying a substantially higher price for the feed gas used. The total capital expended at that point is $211 billion. In year 11 (with no more plants being constructed) the net income after taxation is $181.1 billion. These large amounts of capital in one form or another have been obtained with a maximum initial capital draw in the first year of only $5.7 billion.

The second Example is aimed at production of methanol only in Gas-To-Methanol plants. The use of the methanol is for fuel in gas turbines, most likely for generating electricity.

The plant size is assumed to be 4,500 MTPD methanol. Its cost under the in series construction is assumed to be reduced to $210 million. Feed costs are at $15/metric ton (MT) value is taken at $85/MT. Transport cost of the large amounts of methanol is assumed to be $5/MT. All cost amounts in the table are in billions of dollars.

### EXAMPLE [B]

| | YEAR | | | | | | | | | | |
|---|---|---|---|---|---|---|---|---|---|---|---|
| | 1 | 2 | 3 | 4 | 5 | 6 | 7 | 8 | 9 | 10 | 11 |
| Number of plants | 40 | 15 | 20 | 25 | 40 | 50 | 80 | 100 | 100 | 100 | 0 |
| Cost/Yr Billion $ | 8.4 | 3.2 | 4.2 | 5.3 | 8.4 | 10.5 | 16.8 | 21.0 | 21.0 | 21.0 | 0 |
| Billion $ PROD. | 2.6 | 6.3 | 8.6 | 11.5 | 15.8 | 21.8 | 30.4 | 42.2 | 55.4 | 68.6 | 75.2 |
| Billion $ FEED | 0.6 | 1.5 | 2.0 | 2.7 | 3.7 | 5.1 | 7.1 | 9.9 | 13.0 | 16.1 | 17.7 |
| 15% CAP. DRAW | 1.1 | 1.2 | 1.2 | 1.1 | 1.2 | 1.0 | 1.1 | 0.8 | 0 | 0 | 0 |
| TAX 35% | 0.02 | 0.9 | 1.3 | 2.0 | 2.8 | 4.1 | 5.8 | 8.3 | 11.4 | 14.2 | 16.2 |
| Net Income | 0.9 | 2.7 | 4.0 | 5.7 | 8.1 | 11.6 | 16.4 | 23.2 | 31.0 | 38.3 | 41.3 |
| Cap. Draw | 7.5 | 7.9 | 8.1 | 7.6 | 7.9 | 6.8 | 7.3 | 5.1 | −4.9 | −22.2 | −63.5 |

Compared to Example A the growth in net income is much slower. Money in hand after 10 years is $22.2 billion, and as before all plants are paid for and no capital draw is left. It is still a satisfying operation.

With the large growth expected for the GTL plants generating Fischer-Tropsch hydrocarbons, it stands to reason, that at some time a large extra number of these plants might become less attractive. In that case a combination of a small additional hydrocarbon production with a large methanol production might be attractive. This case is illustrated in Example [C]. where it is assumed still a minor amount of HC plants can be produced, but the capital generating power of these HC plants then is used to stimulate methanol growth.

### EXAMPLE [C]

| | YEAR | | | | | | | | | | |
|---|---|---|---|---|---|---|---|---|---|---|---|
| | 1 | 2 | 3 | 4 | 5 | 6 | 7 | 8 | 9 | 10 | 11 |
| Number HC PLTS | 50 | 0 | 0 | 0 | 0 | 0 | 0 | 0 | 0 | 0 | 0 |
| Billion $ HC PLTS | 11.7 | 0 | 0 | 0 | 0 | 0 | 0 | 0 | 0 | 0 | 0 |
| Number MeOH PLTS | 0 | 55 | 75 | 100 | 100 | 100 | 100 | 100 | 100 | 100 | 0 |
| Billion $MeOH PLTS | 0 | 11.6 | 15.8 | 21.0 | 21.0 | 21.0 | 21.0 | 21.0 | 21.0 | 21.0 | 0 |
| TOTAL PLTS/YR | 11.7 | 11.6 | 15.8 | 21.0 | 21.0 | 21.0 | 21.0 | 21.0 | 21.0 | 21.0 | 0 |
| Billion $ PROD. | 7.8 | 19.3 | 27.8 | 39.4 | 52.6 | 65.8 | 79.0 | 92.2 | 105.4 | 118.6 | 125.2 |
| Billion $ FEED | 0.4 | 1.6 | 3.6 | 6.3 | 9.4 | 12.5 | 15.6 | 18.7 | 21.8 | 25.0 | 26.5 |
| 15% CAP DR. | 1.1 | 1.1 | 1.0 | 0.6 | 0 | 0 | 0 | 0 | 0 | 0 | 0 |
| TAX 35% | 1.8 | 5.0 | 6.8 | 9.3 | 12.3 | 15.1 | 17.9 | 20.7 | 23.5 | 26.3 | 28.4 |
| NET INCOME | 4.6 | 11.6 | 16.5 | 23.2 | 30.9 | 38.2 | 45.5 | 52.8 | 60.1 | 67.4 | 70.2 |
| CAP. DRAW | 7.2 | 7.1 | 6.4 | 4.2 | −5.7 | −22.9 | −47.4 | −79.2 | 118.2 | −164.6 | −234.8 |

This Example clearly shows a stimulation of the methanol plant amounts by just one year of hydrocarbon production. Other combinations will most likely happen in practicing the teachings of this Patent Application.

1. In a Natural Gas To Liquid product (GTL) conversion process in which natural gas is first converted into a synthesis gas, said synthesis gas thereafter being converted into an organic liquid product, an improvement comprising:
employing a multiplicity of individual vessel-based GTL conversion units which are of a substantially identical design in so far as each unit converts natural gas into a synthesis gas.

2. The process of claim 1, wherein each of said GTL conversion units are designed to produce from about 1,000 to about 6,000 metric tons per day (MTPD) of organic liquid product.

3. The process of claim 2, wherein the number of GTL conversion units is at least 10.

4. The process of claim 3, wherein the number of GTL conversion units is at least 20.

5. The process of claim 4, wherein the number of GTL conversion units is at least 50.

6. The process of claim 1, wherein at least some of the multiplicity of GTL conversion units produce Fischer-Tropsch type hydrocarbons.

7. The process of claim 1, wherein at least some of the multiplicity of GTL conversion units are designed to produce methanol or dimethyl-ether as the organic liquid products.
